(19) United States
(12) Patent Application Publication
(10) Pub. No.: US 2018/0092788 A1
(43) Pub. Date: Apr. 5, 2018

(54) ANTIALLERGEN PATCH

(71) Applicant: The Swatch Group Research and Development Ltd, Marin (CH)

(72) Inventors: Nicolas FRANCOIS, Neuchatel (CH); Erik Jan FRENKEL, Kerzers (CH)

(73) Assignee: The Swatch Group Research and Development Ltd, Marin (CH)

(21) Appl. No.: 15/715,649

(22) Filed: Sep. 26, 2017

(30) Foreign Application Priority Data
Oct. 4, 2016 (EP) 16192207.5

(51) Int. Cl. A61F 13/84; G04B 37/00; A44C 5/00

(52) U.S. Cl. CPC A61F 13/8405; A61F 2013/15048; A44C 5/0053; G04B 37/005

(57) ABSTRACT

The invention relates to an antiallergen patch comprising at least a first, adhesive layer having a first surface intended to fasten the patch to an article and a second surface to which a second, continuous allergen-proof barrier layer is attached, a third layer made of a breathable material, designed to absorb water vapour and dry quickly, said third layer being intended to be in contact with the skin of a person wearing the article, and a fourth, bonding layer, which is placed between the second and third layers and secures said second and third layers to one another.

Such an antiallergen patch may be placed on a watch case back or on a bracelet.

This application claims priority from European patent application No. 16192207.5 filed Oct. 4, 2016, the entire disclosure of which is hereby incorporated herein by reference.

### FIELD OF THE INVENTION

The invention relates to an antiallergen patch comprising at least a first, adhesive layer having a first surface intended to fasten the patch to an article and a second surface to which a second, continuous allergen-proof barrier layer is attached.

### BACKGROUND TO THE INVENTION

Such a patch is known and is described, for example, in patent EP 0 038 793. It describes an object made of a flexible material, such as a watch strap, carrying a patch formed of a continuous barrier layer preventing allergens and/or irritants from passing from the material onto and into the skin of the wearer, and of an adhesive layer. The barrier layer can take the form of a matrix loaded with metal particles or a metal layer made of aluminium or silver. The thickness of the metal layer is between 150 μm and 500 μm, in particular from 200 μm to 350 μm, such that the patch is relatively thick and not very flexible. Moreover, in the case of a metal layer, this is in direct contact with the skin, which can be unpleasant for the person wearing the strap. In the case of a barrier layer comprising metal particles, the barrier layer is not totally protected from the migration of nickel ions, for example, which can be found for instance in the material of a watch case back. Consequently, this type of patch described in patent EP 0 038 793 for being fastened to a leather watch strap is not suitable for being fastened to the metal back of a watch case, for example.

A variant of the patch described in EP 0 038 793 has been marketed and also comprises an adhesive layer and a barrier layer. Said barrier layer is leather coated with a polyurethane layer acting as a barrier layer. In this case too, the patch produced has a very great total thickness of more than 2 mm. Because of this, when this patch is used on a watch case back, the great thickness of the patch leads to aesthetic problems when worn: the watch is too high on the wrist, and the patch is visible when the watch is worn. What is more, wearing it can become uncomfortable. Furthermore, the leather used is chrome-tanned leather, which represents a potential risk of free chromium migration, leading to another risk of allergic reaction if the wearer is allergic to chromium.

### SUMMARY OF THE INVENTION

The invention has the particular aim of mitigating the various disadvantages of known antiallergen patches.

More specifically, one aim of the invention is to provide an antiallergen patch having properties of a totally allergen-proof barrier while combining properties of being comfortable to wear.

To this end, the present invention relates to an antiallergen patch comprising at least a first, adhesive layer having a first surface intended to fasten the patch to an article and a second surface to which a second, continuous allergen-proof barrier layer is attached.

According to the invention, the antiallergen patch comprises a third layer made of a breathable material, designed to absorb water vapour and dry quickly, said third layer being intended to be in contact with the skin of a person wearing the article, and a fourth, bonding layer, which is placed between the second and third layers and secures said second and third layers to one another.

Thus, the patch has an outer layer that is comfortable to wear, intended to be in contact with the skin of the person wearing the article, able to provide the skin with the breathability it requires, while ensuring a totally allergen-proof barrier.

The present invention also relates to an article intended to be worn by a user in contact with the skin, on which article an antiallergen patch as defined above is positioned such that it can be in direct contact with the skin of the user. Such an article is, for example, a watch case comprising a case back on which the antiallergen patch is positioned, a bracelet, a piece of jewellery, or a piece of wearable electronic equipment intended to be in contact with the skin of the user.

The present invention also relates to a method for manufacturing an antiallergen patch as defined above, comprising the steps of:
attaching a second, barrier layer to a first, adhesive layer
attaching a third layer made of a breathable material to the second, barrier layer by means of a fourth, bonding layer placed between said second, barrier layer and said third layer.

### BRIEF DESCRIPTION OF THE DRAWINGS

Other special features and advantages will become clear from the following non-restrictive description, given for information, with reference to the accompanying drawings, in which:

FIG. 1 is a schematic sectional view of a patch according to the invention positioned on the back of a watch case worn by a user.

### DETAILED DESCRIPTION OF THE PREFERRED EMBODIMENTS

The present invention relates to an antiallergen patch intended to be fastened to an article worn by a user in contact with the skin and designed to act as a barrier to allergens present in the article by preventing the allergens present in the material of the article from passing onto and into the wearer's skin. In the following description, such an article is a watch case comprising a watch case back on which the antiallergen patch is positioned. The article may also be a bracelet or any other piece of jewellery or wearable electronic equipment that may come into direct contact with the user's skin. The back of a watch case may comprise, for example, nickel or cobalt, which can be allergenic substances and risk causing allergies when they migrate to the surface of the case back and come into contact with the user's skin. It is also possible that the sweat of some users in regular, direct contact with the metal of the case back could corrode it and release allergens.

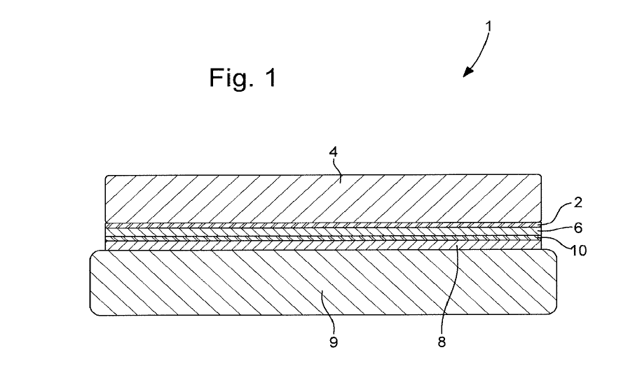

With reference to FIG. 1, the antiallergen patch 1 comprises a first, adhesive layer 2 having a first surface intended to fasten the patch 1 to the back of a watch case 4 and a second surface that is secured to a second, continuous barrier layer 6 that is proof against allergens such as nickel or cobalt, which may be present in the material of the back of the watch case 4.

According to the invention, the patch 1 also comprises a third layer 8 made of a breathable material, designed to absorb the water vapour produced on the wearer's skin 9 and to dry quickly, said third layer 8 being intended to be in contact with the person wearing the article, and therefore constituting an outer layer. The second 6 and third 8 layers are secured to one another by means of a fourth, bonding layer 10 placed between said second 6 and third 8 layers.

Preferably, the third layer 8 may be made of ultrafine nonwoven polyamide microfibres, entangled together for example by needling and impregnated with polyurethane. Preferably, the microfibres have a diameter of less than 0.1 μm, preferably less than 0.08 μm, and more preferably less than or equal to 0.05 μm.

Such microfibres are breathable, i.e. permeable to water vapour. They allow excellent moisture transfer. Because of this, water vapour such as perspiration is absorbed. Such microfibres are also air-permeable and allow excellent air circulation such that the third layer 8 dries very quickly, thus preventing damp/wet contact with the wearer's skin. It is therefore very comfortable to wear.

According to another variant embodiment, the breathable material of the third layer 8 may be a microporous membrane formed of an ultrafine layer of expanded polytetrafluoroethylene, of the GORE-TEX® type.

Preferably, in particular when the third layer is a microfibre, the second, barrier layer 6 may be a non-allergenic metal or metal-alloy layer, based on titanium or aluminium, for example.

In a particularly preferred manner, the second, barrier layer 6 comprises a thin metallised plastics film. Such a layer is produced for example from a polyester film metallised by evaporation, for example. A film of polyethylene terephthalate (PET) metallised by PACVD (plasma-assisted chemical vapour deposition) may advantageously be used.

According to another variant, the second, barrier layer 6 may comprise a layer of nanostructured polymers using inorganic nanofillers so as to make the layer gas- or ion-diffusion-proof.

According to another variant, in particular when the third layer 8 is a GORE-TEX® membrane, the second, barrier layer 6 may comprise a thin plastics film comprising a ceramic coating. Advantageously, a polyethylene terephthalate (PET) film comprising an aluminium oxide coating may be used. A polyethylene terephthalate film coated with aluminium oxide and a GORE-TEX® membrane have the advantage of being transparent. Because of this, the combination of a second, barrier layer comprising a plastics film having a ceramic coating, and a third layer made of a breathable material formed by a microporous membrane of expanded polytetrafluoroethylene, and more particularly the combination of a barrier layer formed by a polyethylene terephthalate film having an aluminium oxide coating, and a third layer formed by a GORE-TEX® membrane, makes it possible to produce a completely transparent patch, by also choosing a transparent first, adhesive layer and a transparent fourth, bonding layer. The patch according to the invention is thus invisible once put in place, which makes it possible to keep the aesthetics of the article, particularly the watch case back, unchanged.

Advantageously, the thickness of the second, barrier layer 6 is less than 50 μm, preferably less than 20 μm, and more preferably less than or equal to 15 μm. A very fine thickness is produced, particularly by using a metallised plastics film, for which the thickness of the metal layer itself is less than 5 μm. This film makes it possible to produce a particularly flexible patch.

The patch according to the invention has a total thickness of between 0.3 mm and 0.8 mm. It is therefore very thin and can be worn very discreetly under the watch case back.

The second, barrier layer 6 and the third layer 8 are secured to one another by means of the fourth, bonding layer 10, which may advantageously be an strong, irreversible adhesive layer.

Advantageously the first, adhesive layer 2 is based on a repositionable adhesive and comprises a detachable protective film. Such a repositionable adhesive has the advantage of allowing the patch to be removed and repositioned as often as necessary without leaving any residue on the watch case back.

According to a first variant, the repositionable adhesive may be a pressure-sensitive adhesive (PSA) formulated, for example, on the basis of an acrylic resin so as to have strong adhesion or, preferably, on the basis of a silicone resin or any other adhesive free of substances that may cause skin reactions.

According to a second variant, the repositionable adhesive may be a Gecko® Nanoplast® microstructured silicone film.

Advantageously, the third layer 8, particularly when made of microfibre, may comprise a decoration made by laser processing, for example, or by deforming the material of the third layer by hot stamping, for example. Laser processing and hot stamping allow decorations to be created on the third layer while avoiding the addition of colourants that risk causing allergies.

Advantageously, the material of the third layer 8, particularly when microfibres are involved, may be impregnated with a substance chosen from the group comprising antisoiling agents, cosmetic active agents for the skin, antibacterial agents and odour-absorbing agents. Antisoiling agents are, for example, fluorinated silanes that are able to impart a water-repellent effect. Cosmetic active agents for the skin may be chosen in order to strengthen the skin's hydrolipidic film. Antibacterial and odour-absorbing agents may be zeolites functionalised with copper, silver or zinc ions that make it possible to prevent the development of bacteria responsible for skin irritations and unpleasant odours. These substances may be impregnated into the material of the third layer in a bath or by supercritical CO₂ treatment.

The patch according to the invention is manufactured by assembling the various layers described above. The method preferably comprises a step of producing the second, barrier layer 6 by metallising a plastics film, and particularly by metallising a polyethylene terephthalate (PET) film by PACVD (plasma-assisted chemical vapour deposition).

The method may also comprise a step of decorating the third layer by laser processing or hot stamping.

The method may also comprise a step of impregnating the material of the third layer 8, in a bath or by supercritical CO₂ treatment, with a substance chosen from the group comprising antisoiling agents, cosmetic active agents for the skin, antibacterial agents and odour-absorbing agents.

This produces a very thin and very flexible patch, very comfortable to wear, allowing the wearer's skin to be provided with the breathability it requires, while ensuring a totally allergen-proof barrier.

Naturally, the present invention is not limited to the examples illustrated and is subject to numerous variants and modifications that will be obvious to a person skilled in the art.

What is claimed is:

1. An antiallergen patch comprising at least a first, adhesive layer having a first surface intended to fasten the patch to an article and a second surface to which a second, continuous allergen-proof barrier layer is attached, wherein said antiallergen patch comprises a third layer made of a breathable material, designed to absorb water vapour and dry quickly, said third layer being intended to be in contact with the skin of a person wearing the article, and a fourth, bonding layer, which is placed between the second and third layers and secures said second and third layers to one another.

2. The antiallergen patch according to claim 1, wherein the breathable material of the third layer is based on ultrafine nonwoven polyamide microfibres impregnated with polyurethane.

3. The antiallergen patch according to claim 2, wherein the microfibres have a diameter of less than 0.1 μm.

4. The antiallergen patch according to claim 3, wherein the microfibres have a diameter of less than 0.08 μm.

5. The antiallergen patch according to claim 4, wherein the microfibres have a diameter of less than or equal to 0.05 μm.

6. The antiallergen patch according to claim 1, wherein the breathable material of the third layer is a microporous membrane of expanded polytetrafluoroethylene.

7. The antiallergen patch according to claim 1, wherein the second, barrier layer is a metal or metal-alloy layer.

8. The antiallergen patch according to claim 1, wherein the second, barrier layer comprises a metallised plastic film.

9. The antiallergen patch according to claim 1, wherein the second, barrier layer comprises a plastics film comprising a ceramic coating.

10. The antiallergen patch according to claim 6, wherein the second, barrier layer comprises a plastics film comprising a ceramic coating, and the breathable material of the third layer is a microporous membrane of expanded polytetrafluoroethylene.

11. The antiallergen patch according to claim 1, wherein the second, barrier layer comprises a layer of nanostructured polymers.

12. The antiallergen patch according to claim 1, wherein the thickness of the second, barrier layer is less than 50 μm.

13. The antiallergen patch according to claim 12, wherein the thickness of the second, barrier layer is less than 20 μm.

14. The antiallergen patch according to claim 13, wherein the thickness of the second, barrier layer is less than or equal to 15 μm.

15. The antiallergen patch according to claim 1, wherein it has a total thickness of between 0.3 mm and 0.8 mm.

16. The antiallergen patch according to claim 1, wherein the first, adhesive layer is based on a repositionable adhesive and comprises a detachable protective film.

17. The antiallergen patch according to claim 1, wherein the third layer comprises a decoration produced by laser processing or hot stamping.

18. The antiallergen patch according to claim 1, wherein the material of the third layer is impregnated with a substance chosen from the group consisting of antisoiling agents, cosmetic active agents for the skin, antibacterial agents and odour-absorbing agents.

19. An article intended to be worn by a user in contact with the skin, on which article an antiallergen patch is positioned such that it can be in direct contact with the skin of the user, said antiallergen patch comprising at least a first, adhesive layer having a first surface intended to fasten the patch to an article and a second surface to which a second, continuous allergen-proof barrier layer is attached, a third layer made of a breathable material, designed to absorb water vapour and dry quickly, said third layer being intended to be in contact with the skin of a person wearing the article, and a fourth, bonding layer, which is placed between the second and third layers and secures said second and third layers to one another.

20. The article according to claim 19, wherein it is chosen from the group consisting of a watch case comprising a case back on which the antiallergen patch is positioned, a bracelet, a piece of jewelry, and a piece of wearable electronic equipment intended to be in contact with the skin of the user.

21. A method for manufacturing an antiallergen patch comprising at least a first, adhesive layer having a first surface intended to fasten the patch to an article and a second surface to which a second, continuous allergen-proof barrier layer is attached, a third layer made of a breathable material, designed to absorb water vapour and dry quickly, said third layer being intended to be in contact with the skin of a person wearing the article, and a fourth, bonding layer, which is placed between the second and third layers and secures said second and third layers to one another, wherein it comprises the steps of:
attaching a second, barrier layer to a first, adhesive layer
attaching a third layer made of a breathable material to the second, barrier layer by means of a fourth, bonding layer placed between said second, barrier layer and said third layer.

22. The method for manufacturing an antiallergen patch according to claim 21, wherein it comprises a step of producing the second, barrier layer by metallising a plastics film.

23. The method for manufacturing an antiallergen patch according to claim 21, wherein it comprises a step of decorating the third layer by laser processing or hot stamping.

24. The method for manufacturing an antiallergen patch according to claim 21, wherein it comprises a step of impregnating the material of the third layer, in a bath or by supercritical CO₂ treatment, with a substance chosen from the group comprising antisoiling agents, cosmetic active agents for the skin, antibacterial agents and odour-absorbing agents.
